(19) United States
(12) Patent Application Publication
(10) Pub. No.: US 2008/0241924 A1
(43) Pub. Date: Oct. 2, 2008

(54) Antibody-Mediated Induction of Tumor Cell Death

(75) Inventors: Thomas Primiano, Chicago, IL (US); Igor B. Roninson, Cohoes, NY (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Board of Trustees of the University of Illinois, Urbana, IL (US)

(21) Appl. No.: 12/139,027

(22) Filed: Jun. 13, 2008

Related U.S. Application Data
(62) Division of application No. 10/692,303, filed on Oct. 23, 2003.
(60) Provisional application No. 60/485,590, filed on Jul. 8, 2003.
(60) Provisional application No. 60/483,684, filed on Jun. 30, 2003.
(60) Provisional application No. 60/420,963, filed on Oct. 24, 2002.

(51) Int. Cl. C12N 5/06

(52) U.S. Cl. 435/375

(57) ABSTRACT

This invention provides methods and reagents for inducing cell death in tumor cells. The invention provides said reagents relating to inducing tumor cell death that are antibodies to a specific target, L1CAM, and methods for using said antibodies for inducing cell death. Pharmaceutical compositions of the L1CAM antibodies for use in the practice of the methods of the invention are also disclosed.

This application claims priority to U.S. provisional applications, Ser. Nos. 60/420,963, filed Oct. 24, 2002, 60/483,684, filed Jun. 30, 2003 and 60/485,590, filed Jul. 8, 2003, the entire disclosures of each of which are explicitly incorporated by reference herein.

This invention was made with government support under grant CA95727 by the National Cancer Institute/National Institutes of Health. The government has certain rights in the invention.

### BACKGROUND OF THE INVENTION

1. Field of the Invention

This invention relates to methods and reagents for inducing cell death in tumor cells. In particular, the invention relates to inducing tumor cell death by contacting the cells with antibodies to a specific target, L1CAM. The invention provides reagents that are antibodies or L1CAM-specific binding fragments thereof, and methods for using said reagents for inducing tumor cell death by contacting tumor cells with said antibodies or antibody fragments. Methods for using said reagents for treating cancer are also disclosed.

2. Background of the Invention

Cancer remains one of the leading causes of death in the United States. Clinically, a broad variety of medical approaches, including surgery, radiation therapy and chemotherapeutic drug therapy are currently being used in the treatment of human cancer (see the textbook CANCER: Principles & Practice of Oncology, 2d Edition, De Vita et al., eds., J.B. Lippincott Company, Philadelphia, Pa., 1985). However, these methods rarely eliminate all tumor cells in a cancer patient, leaving even successfully-treated patients in remission to live under the threat of recurring primary or metastatic disease. In addition, it is recognized that such approaches continue to be limited by a fundamental lack of a clear understanding of the precise cellular bases of malignant transformation and neoplastic growth. Indeed, frequently the most complete understanding of a cancer phenotype is limited to the identification of specific markers for tumors of different types or tissues of origin. Such markers provide convenient targets for developing anticancer therapies.

One such marker is L1 cell adhesion molecule (L1CAM). L1CAM is a 200-220 kDa type I membrane glycoprotein of the immunoglobulin superfamily normally expressed in neural, hematopoietic and certain epithelial cells (Bateman et al., 1996, EMBO J 15: 6050-6059). L1CAM in neural cells has been implicated in cell motility and neurite outgrowth. In addition, chimeric proteins containing the extracellular domains of L1CAM increase neuronal cell survival in serum-free medium (Chen et al., 1999, J. Neurobiol. 38: 428-39). The specificity of the physiological role of L1CAM for neural cells is suggested by the results of mouse knockout studies. L1CAM-null mice develop to adulthood, but they suffer from defects in neural system development, which resemble clinical syndromes of humans with genetic defects in the L1CAM gene (Kamiguchi et al., 1998, Mol. Cell. Neurosci. 12: 48-55).

Neural cell adhesion molecule L1CAM is involved in signal transduction. L1CAM is expressed primarily in the brain, but its expression has also been seen in some other normal tissues and in several types of cancer, including breast cancer. Overexpression of the cell adhesion molecule L1 is associated with metastasis in cutaneous malignant melanoma (Thies et al., 2002, Eur. J Cancer 38: 1708-1716), but there is as yet no evidence that L1CAM plays any role in cell proliferation. Germ-line mutations in human L1CAM have been associated with neural system abnormalities, and similar neurological disorders have been reproduced in L1CAM-null mice, which show apparently normal development in other respects (Kamiguchi et al., 1998, Mol. Cell. Neurosci. 12: 48-55).

The non-neuronal (shortened) form of L1CAM is highly expressed in melanoma, neuroblastoma, and other tumor cell types, including breast. L1CAM is found not only in membrane-bound form but also in the extracellular matrix of brain and tumor cells. Specifically with regard to cancer, L1CAM was found to be expressed in 17/17 surgical samples of small cell lung cancer, with no apparent correlation with the status of cell proliferation (Miyahara et al., 2001, J. Surg. Oncol. 77: 49-54). L1CAM expression was also shown in a lymphoma cell line to provide a negative correlation with lymphoma growth and metastasis (Kowitz et al. 1993, Clin. Exp. Metastasis 11: 419-429).

Antibodies against L1CAM were shown to have several effects in neural cells, including increased influx of calcium (Itoh et al. 1992, Brain Res. 15: 233-240), increased protein phosphatase activity (Klinz et al. 1995, J. Neurochem. 65: 84-95), and inhibition of L1CAM-mediated cell migration (Izumoto et al., 1996, Cancer Res. 56: 1440-1444). Polyclonal antibodies against L1CAM also stimulated ERK2 kinase activity in L1CAM-expressing NIH 3T3 fibroblasts (Schaefer et al. 1999, J. Biol. Chem. 274: 37965-37973), and injection of an anti-L1CAM antibody into mice blocked lymph node fibroblastic reticular matrix remodeling in vivo (Di Sciullo et al., 1998, J. Exp. Med., 1953-1963). In the context of cancer treatment, a monoclonal antibody against L1CAM has been used to target 1311 isotope selectively into neuroblastoma cells (Hoefnagel et al., 2001, Eur. J. Nucl. Med. 28:359-368, 2001).

The present inventors have identified L1CAM as one of several genes whose inhibition results in cytostatic growth arrest in a human breast carcinoma cell line MDA-MB-231 (as described in co-owned and co-pending U.S. patent application Ser. No. 10/199,820, filed Jul. 17, 2002, incorporated by reference). As described in this patent application, L1CAM-derived genetic suppressor elements (GSEs) induce cytostatic growth arrest in the majority of cells, while a minority of cells display characteristic features of mitotic catastrophe (a major form of cell death in tumor cells, which is potentiated by checkpoint deficiencies characteristic of such cells, as discussed in Roninson et al., 2001, Drug Resistance Updates 4: 303-313), exhibited by the presence of multiple micronuclei and abnormal mitotic figures.

L1CAM is a cell surface protein, which is readily accessible to interaction with antibodies or antibody derivatives. The utility of antibody-based drugs in the treatment of cancer has been clearly demonstrated by the example of Herceptin, a humanized monoclonal antibody against Her2/Neu, which shows significant benefit in the treatment of breast cancer (Harries and Smith, 2002, Endocr. Relat. Cancer 9: 75-85). Despite the evidence from GSE studies that inhibition of L1CAM protein expression is detrimental to tumor cell growth, there is no suggestion in the prior art that unconjugated antibodies that interact with L1-CAM on the cell surface may be of use in the treatment of cancer. Importantly, the effects of L1CAM antibodies in neural cells were reported to stimulate rather than inhibit the effects of L1CAM on signal transduction (Schmid et al., 2000, J. Neuorsci. 20: 4177-4188), suggesting that the effects of such antibodies cannot be interpreted as the inhibition of L1CAM function. In view of the important, unmet need in the art to develop agents specifically cytotoxic for cancer cells, there exists a need in the art to determine whether anti-L1CAM antibodies are useful in treating cancer.

### SUMMARY OF THE INVENTION

The present invention provides methods and reagents for inducing cell death in mammalian tumor cells, particularly human tumor cells. The reagents provided by the invention are antibodies, including polyclonal antisera and more preferably monoclonal antibodies, having an antigenic specificity for human-L1CAM protein. In alternative embodiments, the reagents are L1CAM binding fragments of said antibodies. The invention also provides methods for using said reagents to induce cell death in tumor cells, most preferably human tumor cells. In this aspect, the methods of the invention comprise the step of contacting the tumor cell with an effective amount of an L1CAM-specific antibody for a time and at a concentration sufficient to induce cell death in the tumor cells.

Specific preferred embodiments of the present invention will become evident from the following more detailed description of certain preferred embodiments and the claims.

### DESCRIPTION OF THE DRAWINGS

An understanding of the invention is facilitated by reference to the drawings.

FIGS. 1A through 1C show the effects of anti-L1CAM antibodies on normal and tumor cell cultures. FIG. 1A shows the results of fluorescence-activated cell sorting (FACS) analysis of the binding of L1CAM-specific UJ127 antibody to the surface of the indicated cell lines. FIG. 1B shows the effects L1CAM-specific UJ127 (IgG1) and 5G3 (IgG2a) antibodies on the growth of the indicated cell lines. Cells were grown in the presence of 20 nM of the antibodies or the corresponding isotype controls, native or boiled (b), and counted after 4 days (in triplicates). Each bar represents the mean and standard deviation for the number of cells in the presence of the indicated antibodies relative to the isotype controls. The doubling times for each cell line were as follows: MDA-MB231, 42 hrs; MCF7, 41 hrs; HCT116, 38 hrs; HeLa, 39 hrs; BJ1-hTERT, 50 hrs; 184, 41 hrs; 161, 42 hrs; 48RS, 51 hrs. FIG. 1C shows photomicrographs of antibody-treated cells revealing changes in nuclear morphology of tumor cells exposed for 4 days to 5G3 antibody, heat-inactivated (left) or native (right), photographed after DAPI staining at 400× magnification. MN: micronucleated cells, A: apoptotic cells.

### DETAILED DESCRIPTION OF THE PREFERRED EMBODIMENTS

The invention provides reagents and methods for inducing cell death in tumor cells. The reagents provided by the invention are antibodies having antigenic and immunological specificity for L1CAM, and the methods of the invention comprise the steps of contacting tumor cells, most preferably human tumor cells, with an effective amount of the reagents of the invention for a time and at a concentration sufficient to induce tumor cell death.

The term “mammalian L1 cell adhesion molecule” or “L1CAM” as used herein refers to a protein as disclosed in Dahme et al. (1997, Nat. Genet. 17 346-349) and identified in GenBank under Accession No. NM_{—}000425.2. The term is intended to encompass species of said protein from any mammalian species, most preferably humans. The term is also intended to encompass any species having essentially the same amino acid sequence and substantially the same biological activity as the protein identified by Accession No. NM_{—}000425.2. This definition is intended to encompass natural allelic variations and orthologs of the disclosed L1CAM molecule. The invention provides antibodies that are immunologically reactive to L1CAM, most preferably human L1CAM, or epitopes thereof provided by the invention. In some aspects, the invention provides the antibodies of the invention as polyclonal antisera produced in animals experimentally inoculated with one or a plurality of L1CAM-specific antigens. The antibodies provided by the invention may be raised, using methods well known in the art, in animals by inoculation with cells that express L1CAM, most preferably human L1CAM, or epitopes thereof, cell membranes from such cells, including crude protein preparations, or L1CAM proteins obtained using methods well known in the art, including protein fragments and fusion proteins, particularly fusion proteins comprising epitopes of L1CAM, most preferably human L1CAM, fused to heterologous proteins and expressed using genetic engineering means in bacterial, yeast or eukaryotic cells, said proteins being isolated from such cells to varying degrees of homogeneity using conventional biochemical methods. Synthetic peptides made using established synthetic methods in vitro and optionally conjugated with heterologous sequences of amino acids, are also encompassed in these methods to produce the antibodies of the invention. Animals that are useful for such inoculations include individuals from species comprising cows, sheep, pigs, mice, rats, rabbits, hamsters, goats and primates. Preferred animals for inoculation are rodents (including mice, rats, hamsters) and rabbits. The most preferred animal is the mouse.

Cells that can be used for such inoculations, or for any of the other means used in the invention, include any cell line which naturally expresses L1CAM, most preferably human L1CAM, or epitopes thereof, or more preferably any cell or cell line that expresses L1CAM, most preferably human L1CAM, or any epitope thereof, as a result of molecular or genetic engineering, or that has been treated to increase the expression of an endogenous or heterologous L1CAM protein by physical, biochemical or genetic means. Preferred cells are mammalian cells, most preferably cells syngeneic with a rodent, most preferably a mouse host, that have been transformed with a recombinant expression construct encoding L1CAM, most preferably human L1CAM, or epitopes thereof, and that express the protein therefrom.

The present invention also provides monoclonal antibodies that are immunologically reactive with an epitope derived from L1CAM, most preferably human L1CAM, or epitopes thereof, used after varying degrees of biochemical purification. Particularly useful are soluble fragments of L1CAM, most preferably human L1CAM, or epitopes thereof, including for example genetically engineered species. Such antibodies are made using methods and techniques well known to those of skill in the art. Monoclonal antibodies provided by the present invention are produced by hybridoma cell lines, which are also provided by the invention and are made by methods well known in the art.

Hybridoma cell lines are made by fusing individual cells of a myeloma cell line with spleen cells derived from animals immunized with cells expressing L1CAM, most preferably human L1CAM, or epitopes thereof. The myeloma cell lines used in the invention include lines derived from myelomas of mice, rats, hamsters, primates and humans. Preferred myeloma cell lines are from mouse, and the most preferred mouse myeloma cell line is P3×63-Ag8.653. The animals from whom spleens are obtained after immunization are rats, mice and hamsters, preferably mice, most preferably Balb/c mice. Spleen cells and myeloma cells are fused using a number of methods well known in the art, including but not limited to incubation with inactivated Sendai virus and incubation in the presence of polyethylene glycol (PEG). The most preferred method for cell fusion is incubation in the presence of a solution of 45% (w/v) PEG-1450. Monoclonal antibodies produced by hybridoma cell lines can be harvested from cell culture supernatant fluids from in vitro cell growth; alternatively, hybridoma cells can be injected subcutaneously and/or into the peritoneal cavity of an animal, most preferably a mouse, and the monoclonal antibodies obtained from blood and/or ascites fluid.

Monoclonal antibodies provided by the present invention are also produced by recombinant genetic methods well known to those of skill in the art, and the present invention encompasses antibodies made by such methods that are immunologically reactive with an epitope of L1CAM, most preferably human L1CAM, or epitopes thereof. The present invention also encompasses antigen-binding fragments, including but not limited to Fv, F(ab) and F(ab)′₂ fragments, of such antibodies. Fragments are produced by any number of methods, including but not limited to proteolytic or chemical cleavage, chemical synthesis or preparation of such fragments by means of genetic engineering technology. The present invention also encompasses single-chain antibodies that are immunologically reactive with an epitope of L1CAM, most preferably human L1CAM, made by methods known to those of skill in the art.

The present invention also encompasses an epitope of L1CAM, most preferably human L1CAM, comprised of sequences and/or a conformation of sequences present in the molecule. This epitope may be naturally occurring, or may be the result of chemical or proteolytic cleavage of a molecule and isolation of an epitope-containing peptide or may be obtained by chemical or in vitro synthesis of an epitope-containing peptide using methods well known to those skilled in the art. The present invention also encompasses epitope peptides produced as a result of genetic engineering technology and synthesized by genetically engineered prokaryotic or eukaryotic cells.

The invention also includes chimeric antibodies, comprised of light chain and heavy chain peptides immunologically reactive to L1CAM, most preferably human L1CAM, or epitopes thereof. The chimeric antibodies embodied in the present invention include those that are derived from naturally occurring antibodies as well as chimeric antibodies made by means of genetic engineering technology well known to those of skill in the art.

Anti-L1CAM antibodies are also commercially available, and their use with the methods of the invention is also contemplated.

The invention also provides embodiments of anti-L1CAM antibodies or L1CAM antigen-binding fragments thereof as pharmaceutical compositions. The pharmaceutical compositions of the present invention can be manufactured in a manner that is itself known, e.g., by means of a conventional mixing, dissolving, granulating, dragee-making, levigating, emulsifying, encapsulating, entrapping or lyophilizing processes.

Pharmaceutical compositions for use in accordance with the methods of the present invention thus can be formulated in conventional manner using one or more physiologically acceptable carriers comprising excipients and auxiliaries that facilitate processing of L1CAM-specific antibodies or L1CAM antigen-binding fragments thereof into preparations that can be used pharmaceutically. Proper formulation is dependent upon the route of administration chosen.

Anti-L1CAM antibodies or L1CAM antigen-binding fragments thereof can be provided as salts with pharmaceutically compatible counterions. Pharmaceutically compatible salts can be formed with many acids, including but not limited to hydrochloric, sulfuric, acetic, lactic, tartaric, malic, succinic, phosphoric, hydrobromic, sulfinic, formic, toluenesulfonic, methanesulfonic, nitic, benzoic, citric, tartaric, maleic, hydroiodic, alkanoic such as acetic, HOOC—(CH₂)ₙ-CH₃ where n is 0-4, and the like. Salts tend to be more soluble in aqueous or other protonic solvents that are the corresponding free base forms. Non-toxic pharmaceutical base addition salts include salts of bases such as sodium, potassium, calcium, ammonium, and the like. Those skilled in the art will recognize a wide variety of non-toxic pharmaceutically acceptable addition salts.

For injection, anti-L1CAM antibodies or L1CAM antigen-binding fragments thereof can be formulated in appropriate aqueous solutions, such as physiologically compatible buffers such as Hank's solution, Ringer's solution, or physiological saline buffer. For transmucosal and transcutaneous administration, penetrants appropriate to the barrier to be permeated are used in the formulation. Such penetrants are generally known in the art.

For oral administration, anti-L1CAM antibodies or L1CAM antigen-binding fragments thereof can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, anti-L1CAM antibodies or L1CAM antigen-binding fragments thereof can be dissolved or suspended in suitable liquids, such as fatty oils, liquid paraffin, or liquid polyethylene glycols. In addition, stabilizers can be added. All formulations for oral administration should be in dosages suitable for such administration. For buccal administration, the compositions can take the form of tablets or lozenges formulated in conventional manner.

For administration by inhalation, anti-L1CAM antibodies or L1CAM antigen-binding fragments thereof for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The anti-L1CAM antibodies or L1CAM antigen-binding fragments thereof can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the anti-L1CAM antibodies or L1CAM antigen-binding fragments thereof can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances that increase the viscosity of the suspension, such as sodium carboxymethyl cellulose, sorbitol, or dextran. Optionally, the suspension can also contain suitable stabilizers or agents that increase the solubility of the compounds to allow for the preparation of highly concentrated solutions. Alternatively, the active ingredient can be in powder form for constitution with a suitable vehicle, e.g., sterile pyrogen-free water, before use. The compounds can also be formulated in rectal compositions such as suppositories or retention enemas, e.g., containing conventional suppository bases such as cocoa butter or other glycerides.

In addition to the formulations described previously, anti-L1CAM antibodies or L1CAM antigen-binding fragments thereof can also be formulated as a depot preparation. Such long acting formulations can be administered by implantation (for example subcutaneously or intramuscularly) or by intramuscular injection. Thus, for example, the anti-L1CAM antibodies or L1CAM antigen-binding fragments thereof can be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a co-solvent system comprising benzyl alcohol, a nonpolar surfactant, a water-miscible organic polymer, and an aqueous phase. The co-solvent system can be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system can be varied considerably without destroying its solubility and toxicity characteristics. Furthermore, the identity of the co-solvent components can be varied: for example, other low-toxicity nonpolar surfactants can be used instead of polysorbate 80; the fraction size of polyethylene glycol can be varied; other biocompatible polymers can replace polyethylene glycol, e.g. polyvinyl pyrrolidone; and other sugars or polysaccharides can substitute for dextrose.

Alternatively, other delivery systems can be employed. Liposomes and emulsions are well known examples of delivery vehicles or carriers for hydrophobic drugs. Certain organic solvents such as dimethylsulfoxide also can be employed, although usually at the cost of greater toxicity. Additionally, anti-L1CAM antibodies or L1CAM antigen-binding fragments thereof can be delivered using a sustained-release system, such as semipermeable matrices of solid hydrophobic polymers containing the therapeutic agent. Various sustained-release materials have been established and are well known by those skilled in the art. Sustained-release capsules can, depending on their chemical nature, release the anti-L1CAM antibodies or L1CAM antigen-binding fragments thereof for a few weeks up to over 100 days.

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Pharmaceutical compositions of the anti-L1CAM antibodies or L1CAM antigen-binding fragments thereof of the present invention can be formulated and administered through a variety of means, including systemic, localized, or topical administration. Techniques for formulation and administration can be found in “Remington's Pharmaceutical Sciences,” Mack Publishing Co., Easton, Pa. The mode of administration can be selected to maximize delivery to a desired target site in the body. Suitable routes of administration can, for example, include oral, rectal, transmucosal, transcutaneous, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one can administer the anti-L1CAM antibodies or L1CAM antigen-binding fragments thereof in a local rather than systemic manner, for example, via injection of the compound directly into a specific tissue, often in a depot or sustained release formulation.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

For any anti-L1CAM antibodies or L1CAM antigen-binding fragments thereof used in the method of the invention, the therapeutically effective dose can be estimated initially from cell culture assays, as disclosed herein. For example, a dose can be formulated in animal models to achieve a circulating concentration range that includes the EC₅₀ (effective dose for 50% increase) as determined in cell culture, i.e., the concentration of the test compound which achieves a half-maximal amount of tumor cell death. Such information can be used to more accurately determine useful doses in humans.

It will be understood, however, that the specific dose level for any particular patient will depend upon a variety of factors including the activity of the anti-L1CAM antibodies or L1CAM antigen-binding fragments thereof employed, the age, body weight, general health, sex, diet, time of administration, route of administration, and rate of excretion, drug combination, the severity and extent of the particular cancer undergoing therapy and the judgment of the prescribing physician.

Preferred anti-L1CAM antibodies or L1CAM antigen-binding fragments thereof provided by the invention will have certain pharmacological properties. Such properties include, but are not limited to oral bioavailability, low toxicity, low serum protein binding and desirable in vitro and in vivo half-lives. Assays may be used to predict these desirable pharmacological properties. Assays used to predict bioavailability include transport across human intestinal cell monolayers, including Caco-2 cell monolayers. Serum protein binding may be predicted from albumin binding assays. Such assays are described in a review by Oravcová et al. (1996, J. Chromat. B 677: 1-27). Antibody half-life is inversely proportional to the frequency of dosage of the antibody. In vitro half-lives of anti-L1CAM antibodies or L1CAM antigen-binding fragments thereof may be predicted from assays of microsomal half-life as described by Kuhnz and Gieschen (1998, DRUG METABOLISM AND DISPOSION, Vol. 26, pp. 1120-1127).

Toxicity and therapeutic efficacy of said anti-L1CAM antibodies or L1CAM antigen-binding fragments thereof can be determined by standard pharmaceutical procedures in cell cultures or experimental animals, e.g., for determining the LD₅₀ (the dose lethal to 50% of the population) and the ED50 (the dose therapeutically effective in 50% of the population). The dose ratio between toxic and therapeutic effects is the therapeutic index and it can be expressed as the ratio between LD₅₀ and ED₅₀. Antibodies that exhibit high therapeutic indices are preferred. The data obtained from these cell culture assays and animal studies can be used in formulating a range of dosage for use in humans. The dosage of such antibodies lies preferably within a range of circulating concentrations that include the ED50 with little or no toxicity. The dosage can vary within this range depending upon the dosage form employed and the route of administration utilized. The exact formulation, route of administration and dosage can be chosen by the individual physician in view of the patient's condition. (See, e.g. Fingl et al., 1975, in “The Pharmacological Basis of Therapeutics”, Ch. 1, p. 1).

Dosage amount and interval can be adjusted individually to provide plasma levels of the anti-L1CAM antibodies or L1CAM antigen-binding fragments thereof that are sufficient to induce tumor cell death. Usual patient dosages for systemic administration range from 100-2000 mg/day. Stated in terms of patient body surface areas, usual dosages range from 50-910 mg/m/2 day. Usual average plasma levels should be maintained within 0.1-1000 μM. In cases of local administration or selective uptake, the effective local concentration of the anti-L1CAM antibodies or L1CAM antigen-binding fragments thereof cannot be related to plasma concentration.

The invention also provides methods for inducing cell death in tumor cells, most preferably human tumor cells. As disclosed herein, the methods of the invention can be used to induce cell death in any tumor cell that expresses L1CAM, particular tumor cells from breast cancer, colon cancer, cervical cancer, melanoma, neuroblastoma, small cell lung cancer, lymphoma and other tumor cell types. The methods of the invention are effective for inducing cell death in at least 50%, more preferably 60%, more preferably 70%, more preferably 80%, more preferably 90%, more preferably 95%, more preferably 98%, and more preferably 99% of tumor cells. In preferred embodiments, the inventive methods are practiced using the pharmaceutical compositions of the invention as disclosed herein.

The Examples that follow are illustrative of specific embodiments of the invention, and various uses thereof. They set forth for explanatory purposes only, and are not to be taken as limiting the invention.

### Example 1

### Tumor and Normal Cell Growth Inhibition by Anti-L1CAM Antibodies

Anti-L1CAM antibodies were tested to determine their effect on tumor cell growth. The effects of an anti-L1CAM monoclonal antibodies UJ127 and 5G3 on growth of tumor and normal cells were investigated using MDA-MB231 and MCF-7 breast carcinoma cell lines, HeLa cervical carcinoma (all obtained from the American Type Culture Collection, Manassas, Va.) and HCT116 colon carcinoma lines (a gift from B. Vogelstein, Johns Hopkins Medical Institutions, Baltimore, Md.), telomerase-immortalized hTERT-BJ1 normal human fibroblasts (Clontech, Palo Alto, Calif.), and three cultures of normal human mammary epithelial cells (HMEC): 48RS, 184 (both passage 7) and 161 (passage 9), isolated from mammoplasty (provided by Dr. Martha Stampfer, Lawrence Berkeley National Labs, Berkeley, Calif.). HMEC cultures were grown in MEGM, a serum-free medium containing human epidermal growth factor (at a concentration of 10 ng/ml), insulin (5 μg/ml), hydrocortisone (0.5 μg/ml) and bovine pituitary extract (Cambrex BioScience, Walkersville, Md.). The other cell lines were grown in DMEM supplemented with 10% fetal bovine serum.

All the tumor cell lines as well as the normal hTERT-BJ1, 48RS and 184 cells expressed L1CAM on their surface as determined by FACS analysis, but 161 HMEC cells showed no detectable L1CAM. To determine the effect of anti-L1CAM monoclonal antibodies on cell growth and morphology, 10,000-20,000 cells were plated into each well of 24 well plates, and incubated at 37° C. for 4 days in the presence or absence of the corresponding antibody, with change of antibody-containing media after 2 days. Azide-free monoclonal antibodies to L1CAM from hybridoma 5G3 (IgG2a; BD PharMingen, San Diego), and monoclonal antibody to L1CAM from hybridoma UJ127 (IgG1, NeoMarkers (Fremont, Calif.), as well as non-immune IgG1 and IgG2a controls (NeoMarkers) were added to cell culture media at the final concentration of 20 nM and sterilized with 0.22 micron polysulfone filters. As an additional control, monoclonal antibodies were denatured by heating at 95° C. for 10 minutes in 0.1 ml PBS. Cell growth was measured by counting the cell number (in triplicate) using a Coulter counter.

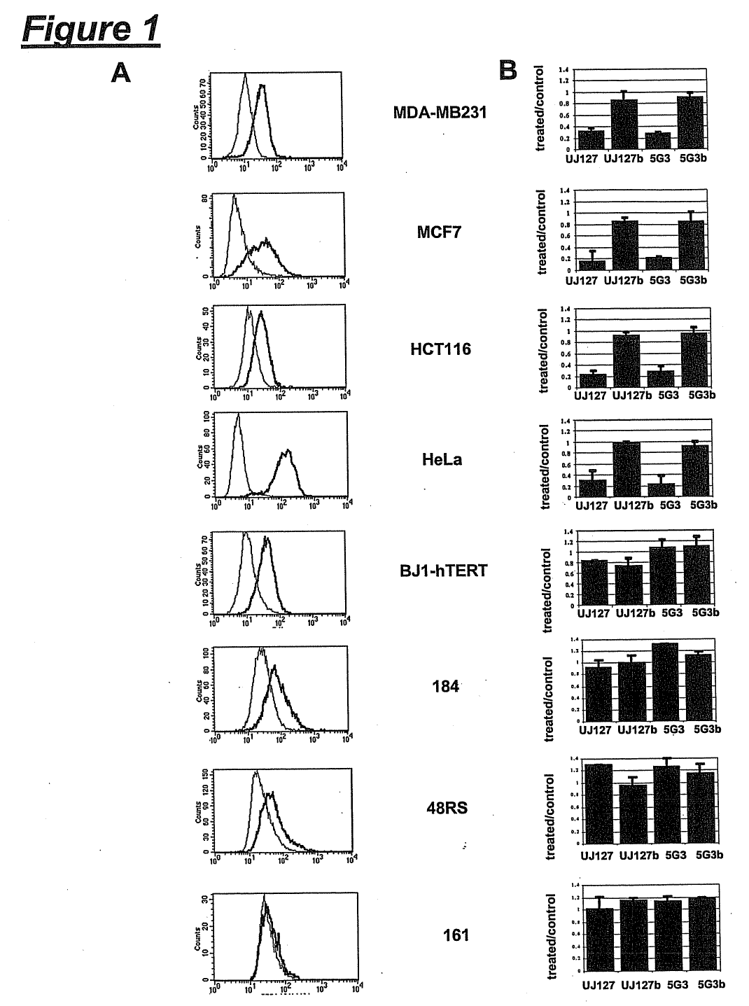

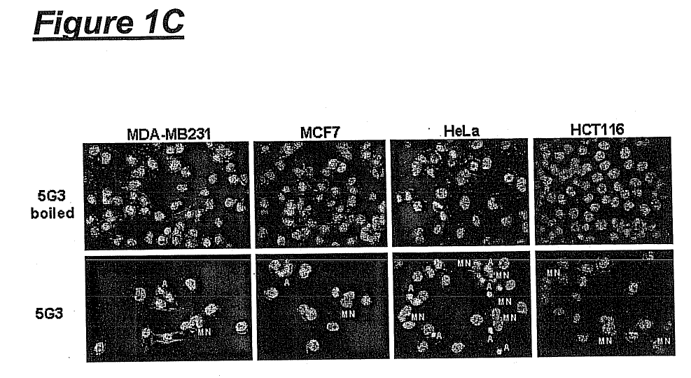

Antibody-treated cells were analyzed by fluorescence-activated cell sorting, carried out using a Becton Dickinson FACSort. All tumor cell lines as well as the normal hTERT-BJ1, 48RS and 184 cells expressed L1CAM on their surface as determined by FACS analysis, but 161 HMEC cells showed no detectable L1CAM. These results are shown in FIG. 1A. The effects of UJ127 and 5G3 antibodies, native or heat-denatured, as well as their corresponding isotype controls, on the growth of all cell lines was investigated for a period of four days, which corresponds to 1.9-2.5 population doublings. These results are shown in FIG. 1B, where the doubling times for each cell line are indicated in the legend. The addition of either UJ127 or 5G3 antibody to the culture media at 20 nM resulted in 3-6 fold decrease in the cell number of all four tumor cell lines relative to their corresponding isotype controls, but the monoclonal antibodies produced little or no growth inhibition in any of the four normal cell cultures. The growth-inhibitory activity of both monoclonal antibodies was abolished by heat denaturation (FIG. 1B).

Similar cytotoxic effects were observed using a rabbit polyclonal anti-L1CAM antiserum, which was obtained from Dr. H. Asou (Keio University, Tokyo, Japan) and which showed a cytotoxic effect at 1:50 dilution.

Nuclear morphology was also observed for all antibody-treated cells. For nuclear morphology analysis, cells were fixed with methanol/acetic acid (5:1), stained with DAPI (5 g/ml in PBS), and examined for blue fluorescence and under phase contrast, using a Leica inverted fluorescence microscope. Microscopic examination of tumor cells remaining on the plate after 4 days of incubation with anti-L1CAM monoclonal antibodies showed the appearance of micronucleated or apoptotic cells, indicative of the induction of cell death (FIG. 1C).

The results obtained with monoclonal antibodies UJ127 and 5G3 and the polyclonal rabbit antiserum suggested that other L1CAM antibodies may have the same effect. Such antibodies can be selected among many existing L1CAM antibodies by the simple assay presented above. Novel monoclonal antibodies and polyclonal antisera against L1CAM can be readily developed by methods well-known in the art, and then tested for the ability to induce tumor cell death. For clinical use, monoclonal antibodies yielding positive results in such assays can be “humanized” through well-known techniques, and antibody derivatives, such as single-chain antibodies and Fab fragments can be readily developed.

It should be understood that the foregoing disclosure emphasizes certain specific embodiments of the invention and that all modifications or alternatives equivalent thereto are within the spirit and scope of the invention as set forth in the appended claims.

What we claim is:

1. A method for inhibiting growth or inducing cell death in a tumor cell, the method comprising the step of contacting the tumor cell with an effective amount of an anti-L1CAM antibody or L1CAM-binding fragment thereof for a time and at a concentration sufficient to inhibit cell growth or induce cell death in the tumor cell.

2. The method of claim 1, wherein the anti-L1CAM antibody is immunologically specific for human L1CAM.

3. The method of claim 1, wherein the tumor cell is a human tumor cell.

4. The method of claim 3, wherein the human tumor cell is a colon cancer cell, a cervical carcinoma cell or a breast cancer cell.

5. The method of claim 1, wherein cell death is induced in at least 50% of the tumor cells.

6. The method of claim 1, wherein cell death is induced in at least 70% of the tumor cells.

7. The method of claim 1, wherein cell death is induced in at least 90% of the tumor cells.
